(19) United States
(12) Patent Application Publication
(10) Pub. No.: US 2023/0397878 A1
(43) Pub. Date: Dec. 14, 2023

(54) MULTIFUNCTIONAL DEVICE AND METHODS FOR IMAGING SURFACE/VOLUME IRREGULARITIES

(71) Applicant: PATTERNOX LTD., Afula (IL)

(72) Inventor: Ofir AHARON, Afula (IL)

(21) Appl. No.: 18/269,955

(22) PCT Filed: Dec. 27, 2021
(86) PCT No.: PCT/IL2021/051539

§ 371 Date: Jun. 28, 2023

Related U.S. Application Data
(60) Provisional application No. 63/130,935, filed on Dec. 28, 2020.

(51) Int. Cl. A61B 5/00; G06T 7/00

(52) U.S. Cl. CPC A61B 5/442; G06T 7/0012; G06T 2207/10152

(57) ABSTRACT

The disclosure relates to systems, methods and programs for generating an image depicting the deformations of a surface and/or volume portion of a subject as a consequence of the surface's and/or volume's structural irregularities (SViR), as a function of density and/or changes in refraction index irregularities and less as a function of color. More specifically, the disclosure relates to a method of imaging a scattering surface and/or volume implementable in a system having a radiation source (RS) operable to illuminate the scattering surface and/or volume portion from different states of illuminations, such as, for example, angular position changes of RS's Poynting Vector (PV), and collect a plurality of images for each state of PV. Due to the effect generated by the altering PV, images experience lateral motion and lateral phase flow as a new tool to reveal SViR.

### BACKGROUND

The present disclosure is directed in general to systems, methods and programs for generating an image of a scattering surface and/or volume portion of a subject depicting the irregularities of the scattering surface and/or volume irregularities (SViR) portion as a function of the scattering surface's and/or volume's density, as well as a function of the changes in refraction index irregularities and less as a function of color. More specifically, the disclosure relates to a method of imaging a scattering surface and/or volume implementable in a system having a radiation source (RS) operable to illuminate the scattering surface and/or volume portion from different states of illuminations, such as, for example, angular (azimuth and/or latitude, and/or distance from the portion of the at least one of: the tissue surface, and the tissue volume along the longitudinal axis Z_{L}) position changes of RS's Poynting Vector (PV) (see e. g., FIGS. 1A-1C), and collect a plurality of images for each state of PV. Due to the effect generated by the skewed illumination position on the SViR of the scattering surface and/or volume portion, each image will have different shadow for each state of illumination (e.g., angle of PV) (see e.g., FIG. 2).

Skin, and mainly skin lesion, is but one example of the scattering surface and/or volume portion. Malignant melanoma is one of the most rapidly increasing cancers in the world. Successful treatment of melanoma depends on early detection by clinicians with subsequent surgical removal of tumors. In recent years, considerable effort has been expended on developing reliable optical methods for characterizing tissue and monitoring changes. Optical scanners for melanoma detection, were approved for marketing. As reported, the 5-year survival rate is 93 to 97% for melanoma detected at an early stage (e.g., Melanoma in-situ), but drops to between 10 and 20% for advanced stage detection, implying that there is a need for accurate diagnostic devices to enable early detection while avoiding unnecessary biopsies.

For example, dermascopes utilizing light or Raman spectroscopy (for signal analysis) to distinguish malignant from benign skin lesions and have shown sensitivities (subjected to physician experience) ranging from 90 to 99%. Other types of dermascopes illuminate the skin at several wavelengths, and measures light scattered back from the skin (with or without polarizers), and use algorithms of color image analysis, combined with a skin disorder database, to provide treatment suggestion. Yet other devices are based on using a handheld, multispectral scanner and computer software to provide dermatoscopic images, dermal and epidermal pathological characteristics, as well as the ability to catalogue, monitor, and compare lesions over time. In a randomized controlled trial involving large number (over 1000) of suspicious pigmented lesions it was found that adding these instruments to best practices resulted in lower agreement, with inter- and intra-observer concordance being very low, even for expert observers' assessment that the lesion was benign, led to a higher proportion of referrals and therefore biopsies.

Despite extensive research in investigating the varied presentations and physical characteristics of melanoma (and other neoplasial pathologies), the clinical diagnostic specificity and selectivity remains suboptimal. Therefore, there is a need for a handheld, portable device that able to provide SViR imaging at early stages of the scattering surface and/or volume portion deformation monitoring changes over time.

### SUMMARY

In certain exemplary implementations, provided herein, are methods of imaging and characterizing a scattering surface and/or volume portion (e.g., a skin portion) implementable in a system having a RS operable to illuminate the scattering surface and/or volume portion from a plurality of radial angles of the RS's PV, and an imaging module configured to capture a Temporal Shadow Image(s) (TSI) from the scattering surface and/or volume portion (e.g., skin lesion) to generate Lateral Motion of Backscattered Light (LMB), Shadow Gradient Pattern Image (SGPI) and lateral phase flow Image (LPFI), thereby depicting the scattering surface and/or volume irregularities (SViR) portion. The SGPI imaging methods refers in an exemplary implementation, to the accumulated movements (or in other words collective changes in the LMB) among the TSIs captured in a given scan. The LPFI is a map of lateral phase flow (direction of the shadow to light movements) while changing PV in a given scan. The shadow-casting effect originates with the skewed collimated or partially collimated illumination of RS's PV. In the context of the disclosure, Collimation is considered collimation between 0 to about 20° (±5°).

In another exemplary implementation, provided herein is a system for in situ imaging of SViR in a skin lesion of a patient in need thereof, comprising: a housing defining longitudinal axis and a proximal base with transparent contact plate (TCP) for positioning over at least a portion of the skin lesion of the patient; at least one RS unit, operably coupled to the housing disposed distal to the TCP, sized and configured to illuminate in a variable manner at predetermined radial angles relative to the longitudinal axis of the housing; an imaging module sized and configured to capture TSIs each one at different angular state (angles: azimuth or altitude) of RS's PV; and optionally a polarizing and/or filter module disposed between the RS unit and the TCP and/or between the TCP and the lens.

In yet another exemplary implementation, the RS in the system is comprised of a plurality of RSs radially coupled to the housing, each RS configured to illuminate at predetermined timings, and wherein the timings configured to illuminate the lesion by varying the RS's PV angles, the system further comprising a communication module configured to maintain communication with a communication network; and a central processing module (CPM), in communication with the imaging module, each of the plurality of RS, an actuator, and the communication module, the CPM further comprises at least one processor in communication with a non-transitory memory storage device storing thereon a processor-readable media with a set of executable instructions, configured when executed, to cause the at least one processor to: using the plurality of RS, illuminate at least one portion of the scattering surface and/or volume (e.g., skin lesion) from a plurality of angles; capture TSI at each illumination angle; using the captured plurality of TSIs, generate LMB, SGPI and LPFI depicting the portion of the scattering surface and/or volume (e.g., skin lesion) SViR And optionally a polarizing and/or filter module disposed between the RS unit and the TCP and/or between the TCP and the lens.

In yet another implementation of the system used to implement the methods disclosed, the RS (either as a single RS, or as comprised of a plurality of RS's) is coupled to a rotatable annulus, the rotatable annulus configured to at least one of: rotate around the longitudinal axis, and translate axially along the longitudinal axis relative to the scattering surface and/or volume portion (e.g., the skin lesion), the rotatable annulus is operably coupled to an actuator, configured to selectively effect at least one of: rotate the annulus, translate the annulus relative to the scattering surface and/or volume portion in parallel with the TCP and around the longitudinal axis, the system further comprising: a communication module configured to maintain communication with a communication network; and a central processing module (CPM), in communication with the imaging module, the at least one RS, the actuator, and the communication module, the CPM further comprises at least one processor in communication with a non-transitory memory storage device storing thereon a processor-readable media with a set of executable instructions, configured when executed, to cause the at least one processor to: actuate the actuator to rotate the annulus. While rotating, capture a plurality of TSIs from the imaging module; using the captured TSIs to generate LMB, SGPI and LPFI depicting the SViR of the scattering surface and/or volume portion.

In yet another exemplary implementation, provided herein is a method of imaging SViR of a scattering surface and/or volume portion of a patient in need thereof, implementable in a system comprising: a housing defining longitudinal axis and a proximal base with a TCP for positioning over the scattering surface and/or volume portion of the patient; at least one RS operably coupled to the housing disposed distal to the base, sized and configured to emit radiation in a variable manner at a predetermined angle relative to the longitudinal axis of the housing; a lens disposed distal to the TCP; an imaging module sized and configured to capture a TSI due to each illumination of the RS; and optionally a polarizing and/or filter module disposed between at least one of: the RS and the TCP, and between the TCP and the lens, the method comprising: positioning the TCP over at least a portion of the skin lesion; illuminating the lesion using the RS from a plurality of radial angles; capturing an image (TSI) at each angle of illumination; and using the captured TSIs to generate LMB, SGPI and LPFI depicting the SViR of the scattering surface ab/or volume portion.

The systems, devices, methods and programs for generating LMB, SGPI and LPFI to characterize and depict SViR of the scattering surface and/or volume portion, and TSI's patterning behavior, will become apparent from the following detailed description when read in conjunction with the drawings, which are exemplary, not limiting, and wherein like elements are numbered alike in several figures.

### BRIEF DESCRIPTION OF THE DRAWINGS

For a better understanding of the methods and systems for SViR detection of a scattering surface ab/or volume portion (e.g., a skin lesion) utilizing LMB, SGPI and LPFI described herein, with regard to the exemplary implementation(s) thereof, reference is made to the accompanying drawings, in which like numerals designate corresponding elements or sections throughout and in which:

FIG. 1A, shows a schematic representation of a first configuration of the system with an observation TCP, with FIG. 1B showing a schematic representation of another configuration of the system with an observation TCP and a prism deflecting the image captured to an imaging module, while FIG. 1C, illustrates plurality of TSIs generated from differences in the RS illumination at various angles (PV angles, PV1 . . . PVn, n-number of PV angle state).

FIG. 2, illustrating signals of two adjacent pixels in an image captured in an exemplary implementation by a digital camera demonstrating lateral intensity movement and a gradient between the pixels—over time or in other words between (and among) TSIs;

FIG. 3, illustrating the intensity movement between two adjacent pixels in a digital camera (over TSIs) following Pixel1 and Pixel2 in FIG. 2, captured at each time PV angle changes; quantified gradient is shown to illustrate the intensity lateral movements between Pixel1 to Pixel2 and vise versa;

FIG. 4A, depicts an image captured by a dermascope of a benign lesion, FIG. 4B, depicting the LMB images captured at 6 different angles, with FIG. 4C depicting the cumulative SGPI of FIG. 4B, and FIG. 4D, depicting the LFP operation on FIG. 4B, providing the SViR of the benign lesion depicted in FIG. 4A;

FIG. 5A, depicts an image captured by a dermascope of an in-situ (stage 0) malignant melanoma, FIG. 5B, depicting the LMB images captured at the same 6 different angles depicted in FIG. 5B, with FIG. 5C depicting the cumulative SGPI of FIG. 5B, and FIG. depicting the LFP operation on FIG. 5B, providing the SViR of the malignant lesion depicted in FIG. 5A: and

FIGS. 6A, and 6B showing a schematic representation of a configuration of the system with pseudo-perpendicular illumination mirror (or semi mirror, or prism) with polarization disc and tilted mirror and a prism deflecting the image captured to an imaging module, without an analyzer in FIG. 6A, and with an analyzer in FIG. 6B.

### DETAILED DESCRIPTION

Provided herein are exemplary implementation(s) of device, systems and methods for the non-invasive characterization of skin lesions. In addition, the device, systems and methods provided herein may be used to diagnose malignancy of the skin lesion, lead to early detection of malignancy and provide for continuous monitoring of changes in the lesions surface and/or volume irregularities (SViR) over time, which may indicate transitions from benign to malignant (atypical, dysplastic, neoplastic and in between). Lateral motions of Backscattered light (LMB), Shadow Gradient Pattern Image (SGPI) and Lateral Phase Flow Image (LPFI) methods are a way to represent or resemble the SViR in none deterministic way. Hence it gives general information on the SViR of a lesion. But enabling to have a follow-up of the SViR can save lives by catching the lesion deterioration into malignancy at early stage.

Dermascopy is a form of noninvasive diagnostic technique that aims to characterize dermatopathology through the visualization of morphological irregularities and their changes that are not discernible by examination with the naked eye. Several lesion scoring systems are typically used to characterize and diagnose skin lesion as an aid to diagnose, characterize, and determine referral or biopsy. Common examples are the ABCD-E rule, the 7-point checklist (weighted and unweighted 7PCL), three-point checklist (3PCL), and the Menzies method. All of these methods relay on irregular color or irregular color distribution, in the ABCD rule; irregular pigmentation and border in the weighted and unweighted 7PCL; the atypical network (referring to a network of increased variability in the color, thickness and spacing of the lines of the network, asymmetrically distributed, and grayscale colored in the 3CPL; or symmetry of pattern structures through the center of the lesion in the Menzies method.

It stands to reason then, that devices, systems and methods for imaging the SViR using LMB, SGPI and LPFI will be advantageous in characterizing the lesion for further diagnosis, since it is not subjected to color but more aiming to the irregularities which are the precursor to pigmentation. SViR represents the degradation from atypical lesion to dysplastic and neoplastic—to be aware to the changes in SViR over time can be life-saving!. Accordingly and in an exemplary implementation, provided herein is a system for in situ characterizing SViR of a skin lesion in a patient in need thereof, comprising: a housing defining longitudinal axis and a proximal base with a TCP for positioning over at least a portion of the skin lesion of the patient; at least one RS (referring to an illumination device comprising one or more light sources, a power supply, and a controller or switch) operably coupled to the housing disposed distal to the base, sized and configured to illuminate in a variable manner at a predetermined angles relative to the longitudinal axis of the housing; a lens disposed distal to the TCP; an imaging module disposed distal to the TCP, sized and configured to capture the TSIs generated by alternations in Poynting Vector (PV, referring to the energy flux density (energy in Jouls per unit area in mm² per unit time in sec.) of the RSs in various angles (in this case); and optionally a polarizing and/or filter module disposed between the RS and the TCP and/or between the TCP and the lens.

In the context of the disclosure, capturing one image for each state of PV will be named Temporal Shadow Image (TSI). First part of the method is to capture multiple TSIs and extract shadow and light lateral movements. Then a computerize process generates a single image of cumulative SGPI of the scatterer's SViR for the scanned portion. PV alternations can also be revealed by monitoring the LPFI— which is the direction of the lateral changes. LMB originate by altering the PV state. The cumulative LMB images are used to generate the SGPI and the LPFI. Thus, SGPI qualitatively depicts the scatterer's SViR. LMB and LPFI are the characteristics due to the information concealed in the movement of light at the camera detector during altering PV (or RS).

In the context of the disclosure, the term “shadow” refers to the partial shadow-casting caused by all optical phenomena in the scattering surface and/or volume portion (reflection, absorption, depolarization . . . etc.) resulting from the skewed illumination (as alternation of PV). As an extreme example, just how the sun projects different shadows upon a tower. One can imagine the tower become more and more transparent (considering the tower as SViR in the air), the shadow starts to be distorted, and if we collect all of these distortions around the various angles, we can conclude the existence of the transparent tower. Also, the term shadow indicates a condition relating to an irradiation region or lack of it, where illumination from an RS can directly reach the region of interest (e.g., a lesion's border), but illumination from other state of PV, or other radial angles of the same RS cannot directly reach the given region because of a SViR partially being opaque.

Likewise, the term “radiation” or “illumination” are used herein to include the entire spectrum of electromagnetic radiation and any radiation useful to interact with the lesion at various depths is consistent with the present disclosure.

In certain configurations, the RS unit can be comprised of a single RS (illuminator) providing collimated illumination onto the surface of the scatterer (as for skin or lesion), and the change in illumination can be executed by manually or automatically (in other words, without user intervention) rotating the housing to provide variable angles (e.g. radial). The collimation of light should be understood broadly in the context of the disclosure, without limiting its scope. The term “collimation” and its derivatives, e.g., “collimated” and the like, generally refers to reducing the divergence of light decoupled from the illuminator(s) in one or more dimensions and/or planes as compared to the case when the divergence of light exiting the illuminator(s) is primarily defined by the broad angular distribution of light propagating in the illuminator by means of the optical properties of the reflectors coupled to the illuminator(s).

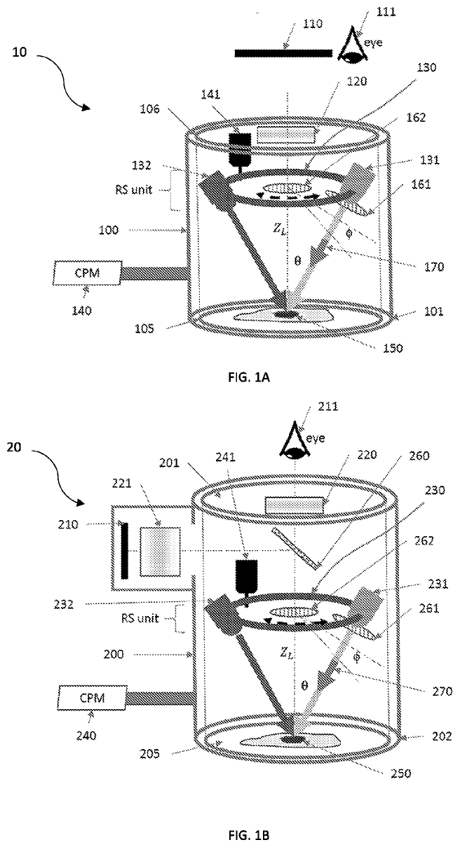

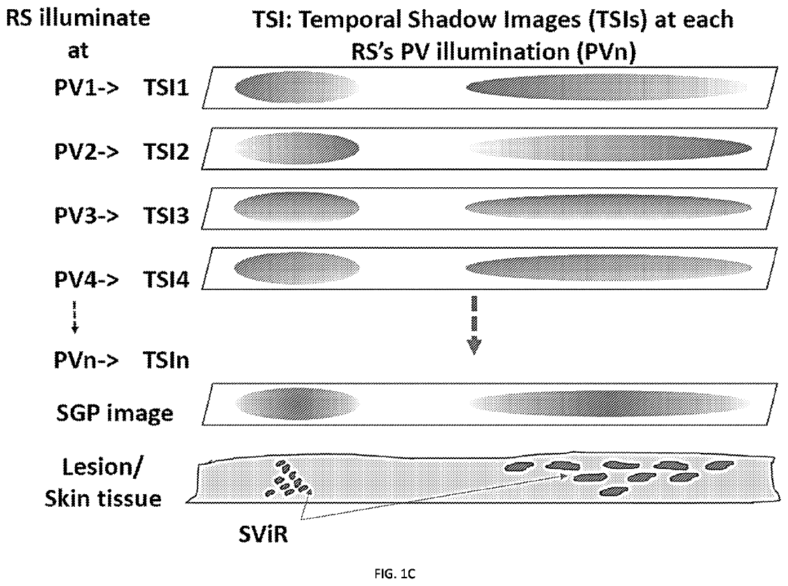

In the context of the disclosure, the term “Radial angle” refers to the angle along the Radial Path around the scanned portion of the lesion (around the longitudinal axis). Additionally, or alternatively, in certain configurations, varying the radial angle can be achieved by a plurality of RS radially (generating variations in PV) coupled to the housing, each RS configured to illuminate at predetermined timing configuration, and wherein the timing configuration configured to illuminate the lesion from varying radial angles. In other words, a series of RS having either the same or different spectrum emission are operably coupled to the housing, forming a circular ring around the longitudinal axis defined by the housing, the RSs (forming the RS complex in one exemplary implementation) being further coupled to an integrated circuit sized, adapted and configured to time and power each light source, thereby providing illumination from a variety of radial angles. In certain implementations, the plurality of RS can be positioned at variable heights relative to the proximal base, thus providing variable illumination angles (θ,ϕ, see e.g., FIG. 1).

In certain other configurations, varying the angle can be achieved by incorporating an annulus within the housing, the annulus being rotatable and coupled to the housing and to an actuator, configured to selectively affect at least one of: rotate the annulus, translate the annulus relative to the skin lesion along the longitudinal axis, the rotatable annulus operational to at least one of: rotate around the longitudinal axis, and translate along the longitudinal axis relative to the skin lesion. In other words, in addition to illuminating the lesion from variable radial angles affected by rotating the annulus, the actuator can further slide the annulus up and/or down relative to the lesion, thus providing different illumination angles thus generating different shadow.

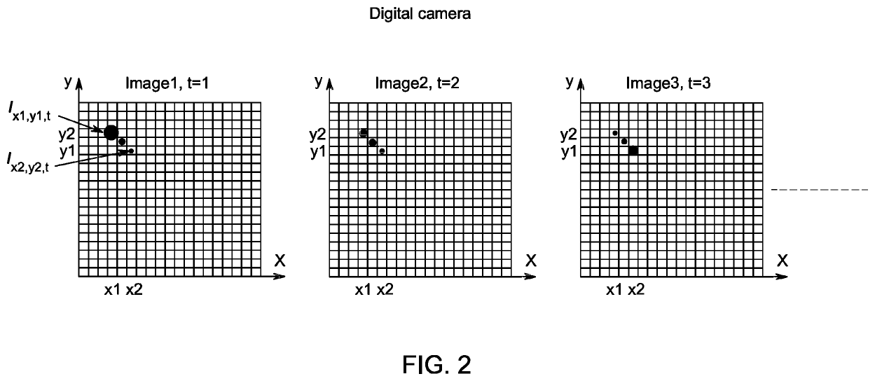

As used herein, the term “actuator” should be understood to encompass a wide variety of devices, motor, including spring, hydraulic, pneumatic, or combinations of spring, hydraulic and pneumatic actuators. The term “actuator” may also encompass a portion of a robotic system configured to apply the requisite actuation motion to the annulus. The actuator (see e.g., FIG. 1A, element 141) is operative to actuate the annulus to rotate relative to the housing. As used herein, the term “actuator” broadly refers to any device used to directly or indirectly move another device (and, thus, is operative to actuate (or, in some situations, rotate) the annulus 130. An actuator can take any suitable form, including, but not limited to, a motor, a linear actuator, a piston, a rack-and-pinion device, and systems that use hydraulic fluids. An actuator “actuates” the annulus by moving it in any suitable manner, including, but not limited to at least one of: rotating, vibrating, and linearly moving the annulus 130. While not required in all implementations and configurations, the actuator can also be operative to track the position of the at least one RS. Furthermore, in some implementations, the actuator actuates the annulus (i.e., actuates in any suitable form of actuation). In other implementations, the actuator translates the annulus up and/or down relative to the proximal base of the housing. Accordingly, the term “actuate” in the claims should not be limited to rotate. Also, actuators other than a motor (such as those listed above) can be used to rotate the first reflective surface. Accordingly, the phrase “actuator operative to rotate” in the claims should not be limited to a motor. The angular changes shall be defined as the changes in latitude and longitude of at least 1 degree (ϕ, θ in FIG. 1-2).

As indicated, the lens present in the housing, can be a removable and be configured to filter visible light spectrum, providing incident light (IL, see e.g., FIG. 1A) at a predetermined wavelength, for example, between about 350 nm and about 1000 nm. The term “filter” refers to a material (e.g., the lens composition) that prevents the transmission of some wavelengths region to go pass through the filter material, regardless of the nature of the prevention mechanism, thus provide additional information on the SViR.

To acquire a TSI of a portion of the scattering surface and/or volume (e.g., skin lesion), it is illuminated in certain configurations with illumination that exhibits the appropriate intensity, spread, wavelength and timing for acquisition. Depending upon the features being imaged (e.g., sort of SViR of a lesion) and the relative angle of the imaging module's (e.g., camera) axis to the features, the type and characteristics of illumination employed can vary widely. For example, some features (e.g. lesion thickness) can be best imaged using low B angle illumination, while other features (e.g. border) are typically best imaged using direct, high Bangle illumination. Likewise, the wavelength and spectrum of illumination can vary depending on the nature of the features—that is, some features are best illuminated in the visible range, while others may be enhanced using infrared (IR) light.

Each RS illuminator can comprise a light-emitting diode (LED), configured to emit electromagnetic radiation at a predetermined wavelength and intensity. The wavelength can be the whole visible spectrum (e.g., incandescent, white LED, or white organic light-emitting diode, WOLED) with a filter lens that will allow a predetermined wavelength to be emitted from the lens. RS can also be controlled to a potentiometer or PWM (pulse width modulator) and be configured to selectively operate as a strobe (in other words, provide flashes of light intensity emission for a short duration).

Likewise, for some applications it is desirable to employ polarized light and/or filter in the light path between RS and lesion and/or between lesion and camera. Certain implementations disclosed can comprise a ring illumination arrangement consisting of one or more rows of that encircle the lens. These illuminators can be external and remote from the camera housing, but “internal” in other configurations, being fixed in place on housing above the TCP. One form of ring illuminator, which surrounds the camera lens, is constructed in certain implementations on a circuit board that contains a predetermined number of lighting elements in one or more circles around the lens. The lighting elements on a typical illuminator are LEDs, but other types of light sources (e.g. xenon strobes, incandescent, laser, fluorescent light source, etc.) can also be employed, either as an alternative to LEDs or in addition to LEDs.

Accordingly, the term RS or “illuminator” as used in the context of the disclosure, shall refer generally to internal or external illuminators that are controlled by the CPM. Also, as used herein, the term “processor” in relation to the imaging module shall refer to one or more processing components that are generally contained within the imaging module unit, but that can be (at least in part) located remote from the imaging module unit enclosure and that carry out the illustrative functions described herein. These functions include control of the RS and settings that are dependent on the RS unit's parameters. Other image processing, camera-control functions and control on the actuator can also be performed by the “processor.”

Furthermore, it is noted that the term “imaging module” as used herein means a unit that includes a plurality of built-in image and/or optic sensors and outputs electrical signals, which have been obtained through photoelectric conversion, as an image, while the term “module” refers to software, hardware, for example, at least one processor, or a combination thereof that is programmed with instructions for carrying an algorithm or method steps as disclosed herein. The modules described herein may communicate through a wired connection, for example, a hard-wired connections, a local area network, or the modules may communicate wirelessly. The imaging module may comprise camera (analog to digital detector) like charge coupled devices (CCDs) or metal-oxide semiconductor (CMOS) or as such. TSI is acquired by the imaging module that can comprise a digital frame of the camera, where the field of view (FOV) can be predetermined by, for example, the camera size with its optics and the axial distance from the lesion surface (the TCP). The term “digital camera” refers to implementations to a digital still camera or a digital video recorder. The digital camera can comprise an image capturing unit or module, a capture controlling module, a processing unit like micro controller, field-programmable gate array (FPGA), or local processor (which can be the same or separate from the central processing module).

The transmission at the various wavelengths is configured each to penetrate the surface of the lesion or skin to different depth, thus providing different TSI and different information. Generally speaking, in the optical and near infra-red (NIR) region the shorter the wavelength the less penetration, hence different shadow in the TSI due to differences in scattering, absorption, refraction and reflection.

As illustrated in FIG. 1B, in certain configuration, the device used in the system disclosed further comprising: a transparent window as TCP coupled to the housing, sized and configured to allow direct observation of the skin lesion by a user (e.g., a physician, care provider, the patient etc.); and a prismatic element (referring to an optical prism, mirror, partial mirror), sized and configured to divert lesions backscattered light toward the imaging module.

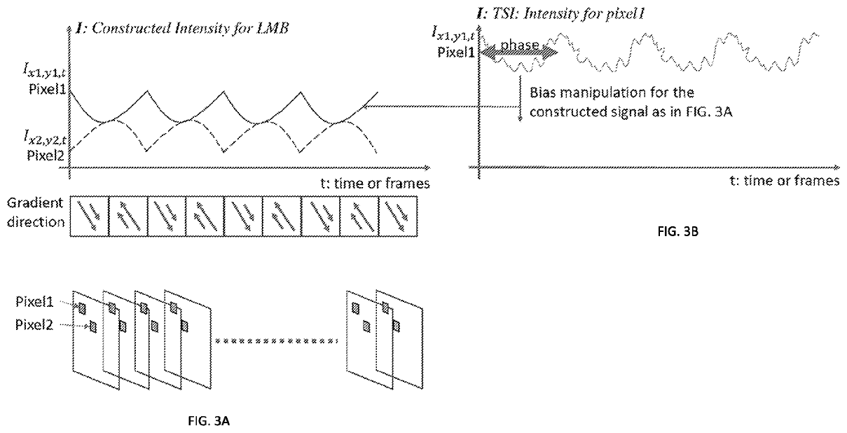

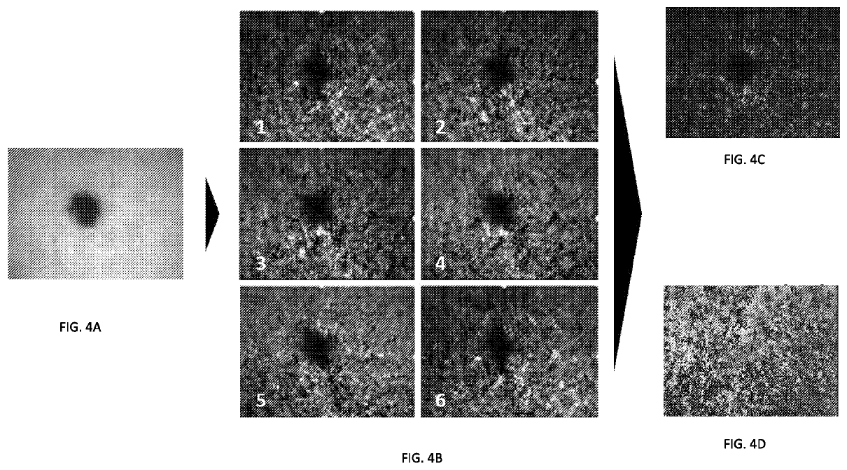

As indicated, the devices used as part of the systems disclosed herein, use an RS unit, comprised of a plurality of RS illuminators having maximum emission wavelength that is the same or different, arranged in certain configurations as a circle operably coupled to at least one of: the housing, the annulus, and around the lens of the imaging module. Controlling the illumination behavior can be done by a switch (as described hereinabove), or at least one processor that is in communication with the imaging module and the CPM. In these implementations, the system can further comprise: a communication module configured to maintain communication with a communication network; and a CPM, in communication with the imaging module, the at least one illuminator, and the actuator. The communication module and the CPM comprises at least one processor in communication with a non-transitory memory storage device storing thereon a processor-readable media with a set of executable instructions, configured when executed, to cause the at least one processor to: using the plurality of RS' illuminators, illuminate the lesion from a plurality of angles; capture an image at each illumination angle, each image providing a TSI (see e.g., FIG. 1C, FIG. 2, FIG. 3, bottom); using the captured plurality of TSIs, calculate the LMB by constructing the signal for each pixel in a periodic way using the signal phase (in every pixel) and eliminating or modifying its bias. (the scan itself don't need to be periodic). It generates the lateral movements (LMB). Then calculate the total cumulative LMB as one image of SGPI and LPFI depict the SViR of the scattering surface and/or volume portion (skin lesion in this example) (see e.g., FIG. 1C bottom and FIGS. 4B-4D).

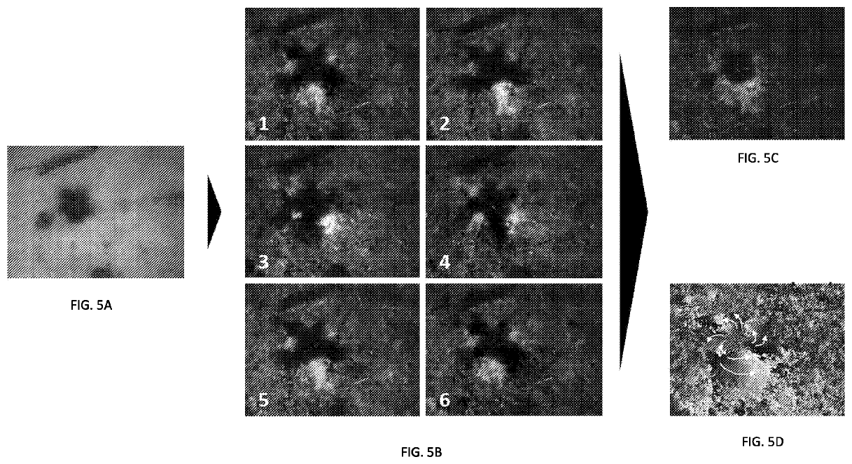

Turning now to FIGS. 4A-D, and 5A-D, where FIG. 4A-C illustrate a study case images of the SViR of a benign lesion as calculated by LMB (FIG. 4B), and SGPI (FIG. 4C), depicting a dermascope-captured lesion in FIG. 4A, being compared with skin cancer FIG. 5A showing a lesion with high surface variability as can be shown in its LMB (FIG. 5B) and its SViR imaging (FIG. 5C). Each of the six images shown in their LMB stems from different state of PV. Nevertheless, the dermoscopy images FIG. 4A and FIG. 5A look very similar, a prominent change in the SViR can show its differences in the underneath deformation (SViR). The benign case (FIG. 4C) revealed to be with random patterns and the skin cancer (FIG. 5C) looks like an explosion from the middle outward. Figures FIG. 4D and FIG. 5D are the LPFI of the lateral phase indicate the lateral movements of the LMB. The directions of the arrows go from dark to bright (phase of −3.1415 to 3.1415).

In the context of the disclosure, the term “random”, is not intended necessarily to denote pure randomness in the theoretical sense. A signal such as the LPFI, does not need to be purely random in order for it to be sufficiently random to be useful. The degree of randomness, or unpredictability, depends on the particular application of use. Therefore, the term “random” will be used herein to indicate a degree of unpredictability or randomness sufficient for use as a predictor of malignancy. Furthermore, and in reference to FIGS. 2, 3A and 3B, the term “unpredictability”, as that term is used herein, is intended to mean that, given a perfect knowledge of the circuit and of the waveform of a signal up to a time “t”, the value of the signal within some limit of precision at time “t+Δt” cannot be predicted. As the odds of a prediction being wrong approach 50% each time a prediction is made, the result can be regarded as a random bit string (captured in the randomness illustrated in FIG. 4D.

Using LMB, SGPI and LPFI methods, physicians will have new tool to make a more educated decision about the state of the tissue. As illustrated in comparing FIGS. 4C, 4D, 5C and, 5D, it is evident that random patterns imply a healthy tissue (or lesion) and directional (or otherwise, non-random) patterns, typically resembling a nimbus cloud or a burst imply dysplasm of the lesion, which may be a precursor of neoplasm (cancer).

As indicated, FIG. 4B shows six LMB images (constructed from TSIs using signal phase and modified bias) at six PV state illustrating the LMB effect of illuminating in various angles of RS (PV angles) on shadow-casting and resulting in the SGPI as shown in FIG. 4C. For healthy tissue the patterns are random compared with FIG. 5B-C which is show distorted lesion by directional patterns. FIG. 5B are six LMB images (constructed from TSIs using signal phase and modified bias) illustrating the LMB effect of illuminating in various angles of RS (e.g., PV angles), its result is the SGPI that indicates directional patterns as shown in FIG. 5C for neoplasm (skin cancer). SGPI as will be further explained is the accumulated changes (lateral gradient and/or phase) between the LMB images, laterally and in time as shown in FIG. 3. Using LMB, SGPI and LPFI methods physicians will have new tool to make a better decision about the state of the tissue. When the pattern is random it mostly implies on a healthy tissue (or lesion) and when the patterns are directional, mostly as nimbus clouds or burst of an explosion it depicts high deformations as in the case of dysplasm or neoplasm of the lesion which may be a precursor of cancer or already cancer.

In an exemplary implementation, the systems disclosed herein are used to implement the methods disclosed. Therefore, provided herein is a method of imaging and characterizing SViR of a scattering surface and/or volume portion (e.g., a skin lesion), implementable in a system comprising: a housing defining longitudinal axis and a proximal base with an TCP (coupled at the proximal base) for positioning over at least a portion of the skin lesion of the patient; at least one RS unit, operably coupled to the housing disposed distal to the base, sized and configured to emit radiation in a variable pattern at a predetermined radial angle relative to the longitudinal axis of the housing; a lens disposed distal to the RS unit; an imaging module sized and configured to capture a shadow pattern (due to phase extraction and bias elimination from TSIs to generate LMB images, FIG. 3B) from the transmission of the RS unit; and optionally a polarizing module disposed between the RS and the lens, the method comprising: positioning the TCP over at least a portion of the skin lesion; radiating the lesion using the RS unit from a plurality of radial angles; capturing an image at each radial radiation angle or polarization, each image providing a TSI; and using the captured images, generating LMB, SGPI and LPFI that depict the skin lesion's SViR. In certain implementations, where the RS unit comprises a plurality of illuminators (with the same and/or different wavelengths), the at least one of: the predetermined angle of at least one illuminator, and the distance between the RS unit and the skin lesion (in other words, the translation along the longitudinal axis (see e.g., FIGS. 1A, 1B), is selectively variable, and the method further comprises capturing a plurality of images at the same radial angle, however, at different axial distance from the skin lesion, and/or different illuminator angle θ (see e.g., FIG. 1A, 1B).

In an exemplary implementation, the step of generating the LMB, SGPI and LPFI of the skin lesion, comprises: generating a plurality of images (or a clip), each image (or frame) being of a single TSI per rotation step at a given axial distance (e.g., along Z_{L}) over the skin lesion; combining the plurality of TSI's to form LMB (see e.g., FIG. 2), SGPI (see e.g., FIG. 4C, 5C) and LPFI (See e.g., FIGS. 4D, 5D) depicting the SViR of the scattering surface ab/or volume portion (for example, the skin lesion) associated with the skin lesion irregularities. In other words, the cumulative SGPI is generated by extracting the LMB from TSI's changes over the frames (or image, each frame is actually a TSI at different PV state of illumination), (angles: azimuth or altitude), see e.g., FIGS. 1A, 1B) during the capturing of plurality of images (TSIs), the changes can be interpreted using in an exemplary implementation, the phase changes of the signal for each pixel and its bias (see e.g., FIGS. 3A, 3B), cumulative lateral gradient, spatial derivatives or mathematical transforms (spatial and periodic) to analyze the signals of the TSIs and LMB. The cumulative lateral movements in the LMB are determined to be SGPI and is associated with the characteristic SViR of skin lesion as shown in FIG. 1C, bottom (and FIGS. 4C, 5C).

A more complete understanding of the components, methods, and devices disclosed herein can be obtained by reference to the accompanying drawings. These figures (also referred to herein as “FIG.”) are merely schematic representations based on convenience and the ease of demonstrating the present disclosure, and are, therefore, not intended to indicate relative size and dimensions of the devices or components thereof, their relative size relationship and/or to define or limit the scope of the exemplary implementation(s). Although specific terms are used in the following description for the sake of clarity, these terms are intended to refer only to the particular structure of the exemplary implementation(s) selected for illustration in the drawings, and are not intended to define or limit the scope of the disclosure. In the drawings and the following description below, it is to be understood that like numeric designations refer to components of like function. Likewise, cross sections are referred to on normal orthogonal coordinate system having XYZ axis, such that Y axis refers to front-to-back, X axis refers to side-to-side, and Z axis refers to up-and-down. Coordinates illustrating the RS movements are cylindrical, azimuth −θ, latitude −ϕ, height −Z_{L}.

Turning now to FIG. 1A illustrating a schematic representation of a first configuration of system 10 with camera as an optional replacement of the direct looking by eye, while FIG. 1B showing a schematic representation of another configuration of basic system 20 as in FIG. 1A but with a camera coupled radially, using deflector as a prism, partial mirror, or a mirror. As illustrated in FIG. 1A, Basic system 10 comprises housing 100 defining longitudinal axis Z L and proximal base 101 with a TCP 105 for positioning over at least a portion of the skin lesion 150 of the patient; at least one RS unit. In the drawings one can find an example of two RSs 131, 132 but it can be any number of RS operably coupled to the housing 100 disposed distal to the proximal base 101, sized and configured to illuminate in a variable manner at a predetermined angle (latitude θ, azimuth ϕ, also can be illustrated by RS's PV direction 170) relative to the longitudinal axis Z_{L} of housing 100; lens 120 disposed distal to RS unit 131, 132; imaging module 110 disposed distal to RS unit 131, 132, sized and configured to capture a TSIs at every angle of RS illumination 131, 132; and optionally a polarizing or filter module 161 disposed between RS unit 131, 132 and TCP and/or as indicates 162 between TCP and imaging module 110 or eye of user 111 above the Transparent Observation Plate (TOP 106). Also illustrated in FIG. 1A, is central processing module (CPM 140), in communication with imaging module 110, the at least one illuminator (together forming an implementation of RS unit 131, 132 in this example), actuator 141, and the communication module (not shown, part of CPM 140), CPM 140 further comprises at least one processor (not shown, part of CPM 140), in communication with a non-transitory memory storage device (not shown, part of CPM 140), storing thereon a processor-readable media with a set of executable instructions, configured when executed, to cause the at least one processor to carry out the method steps disclosed herein. Also shown is annulus 130, of an exemplary implementation, coupled to housing 100 by being partially inserted in radial channel (not shown).

Turning now to FIG. 1B, illustrating a schematic representation of another exemplary implementation of system 20 with observation TOP 201. Exemplary implementation illustrates system 20 as comprising: housing 200 defining longitudinal axis Z_{L} and proximal base 202 with an TCP 205 for positioning over at least a portion of the skin lesion 250 of the patient; In the drawings one can find an example of two RSs 231, 232 but it can be any number of RS operably coupled to the housing 200 disposed distal to the proximal base 202, sized and configured to illuminate in a variable manner at a predetermined angle (latitude θ, azimuth ϕ, also can be illustrated by RS's Poynting Vector direction 270) relative to the longitudinal axis Z_{L} of housing 200; lens 220 disposed distal and transverse to RS unit (231, 232); imaging module 210 disposed distal to lens 221, sized and configured to capture a TSIs at every angle of RS illumination (231, 232); and optionally a polarizing module and/or filter 261, 262 disposed between RS unit (231, 232) and TCP and/or the TCP and lens 221, 220. System 20 further comprises: transparent observation plate (TOP) 201 situated above the prism inside housing 200, sized and configured to allow direct observation of the skin lesion 250 by the user 211; and prismatic element or mirror or partial mirror 260, sized and configured to divert the backscattered light from skin lesion 250 to lens 220 and 221. Also illustrated in FIG. 1B, is central processing module (CPM 240), in communication with imaging module 210, the at least one illuminator (231, 232 in this example together forming an implementation of RS unit), actuator 241, and the communication module (not shown, part of CPM 240), CPM 240 further comprises at least one processor (not shown, part of CPM 240), in communication with a non-transitory memory storage device (not shown, part of CPM 240), storing thereon a processor-readable media with a set of executable instructions, configured when executed, to cause the at least one processor to carry out the method steps disclosed herein. Also shown is annulus 230, of an exemplary implementation, coupled to housing 200 by being partially inserted in radial channel (not shown).

The device can further comprise a central processing module (CPM). According to various exemplary implementation(s) disclosed herein, the CPM can comprise a processing unit (typically including an arithmetic logic unit and a control unit) and a memory (also known as “registers,” or/and Read Only Memory (ROM) and/or cache memory). The CPM may also comprise a non-transitory memory device, storing thereon an operating system and/or any further executable instructions and/or data configured to affect the function of the components described herein, according to the methods provided. The non-transitory memory device can be implemented within the CPM or externally. For example, the CPM may share a single memory coupled to a bus. As used herein, the term “memory” refers to any type of long term, short term, volatile, nonvolatile, or other storage devices and is not limited to any particular type of memory or number of memories, or type of media upon which memory is stored.

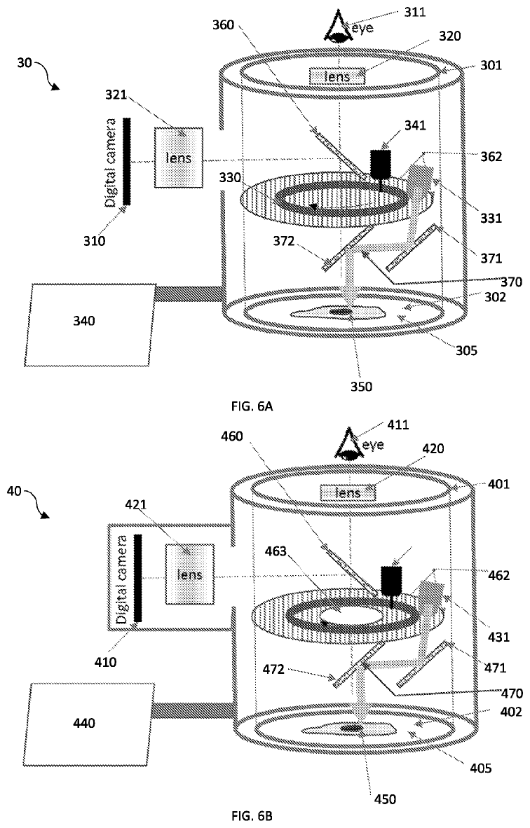

Two more system configurations are illustrated in FIGS. 6A, 6B, showing in FIG. 6A(6B) another exemplary implementation of system 30 with observation TOP 301 (401). Exemplary implementation illustrates system 30 (40) as comprising: housing 300 (400) defining longitudinal axis Z_{L} and proximal base 302 (402) with TCP 305 (405) for positioning over at least a portion of the skin lesion 350 (450) of the patient; At least one RSs 331 (431) is illustrated, operably coupled to the housing 300 (400) disposed distal to the proximal base 302 (402), sized and configured to illuminate in a variable manner at a pseudo-vertical angle (latitude (θ≈80°-89°), azimuth ϕ, as illustrated by RS's 331 (431) deflected Poynting Vector direction 370 (470) relative to the longitudinal axis Z_{L} of housing 300 (400) using at least one of a mirror, half-mirror, and prism arrangement 371,372 (471, 472) to modulate latitude angle θ, making it pseudo-vertical (in other words, about 90°). System 30 (40) also comprise lens 320 (420) disposed distal and transverse to RS unit 331 (431), with imaging module 310 (410) disposed distal to lens 321 (421), sized and configured to capture a TSIs at every pseudo-vertical angle θ of RS illumination 331 (431); and optionally a polarizing module and/or filter 361, (461) disposed between RS unit (331, 431) and TCP 305 (405) and/or an optional polarizing module and/or filter 362 (462) between the TCP 305 (405) and lens 321 (421). System 30 (40) further comprises: transparent observation plate (TOP) 301 (401) situated above prism (or semi-mirror, or mirror) 360 (460) inside housing 300 (400), sized and configured to allow direct observation of the skin lesion 350 (450) by the user 311 (411), sized and configured to divert the backscattered light from skin lesion 350 (450) to lenses 320 (420) and 321 (421) whereby system 40, further comprises analyzer 463. Also illustrated in FIGS. 6A, 6B, is central processing module CPM 340 (440), in communication with imaging module 310 (410), the at least one illuminator 331 (431), actuator 341 (441), and the communication module (not shown, part of CPM 340 (440)), CPM 340 (440) further comprises at least one processor, in communication with a non-transitory memory storage device, storing thereon a processor-readable media with a set of executable instructions, configured when executed, to cause the at least one processor to carry out the method steps disclosed herein. Also shown is annulus 330 (430), of an exemplary implementation of system 30 (40), coupled to housing 300 (400) by being partially inserted in radial channel (3000, (4000), not shown)

All ranges disclosed herein are inclusive of the endpoints, and the endpoints are independently combinable with each other. “Combination” is inclusive of blends, mixtures, alloys, reaction products, and the like. Furthermore, the terms “first,” “second,” and the like, herein do not denote any order, quantity, or importance, but rather are used to denote one element from another. The terms “a”, “an” and “the” herein do not denote a limitation of quantity, and are to be construed to cover both the singular and the plural, unless otherwise indicated herein or clearly contradicted by context. The suffix “(s)” as used herein is intended to include both the singular and the plural of the term that it modifies, thereby including one or more of that term (e.g., the illuminator(s) includes one or more illuminator or emitter of radiation). Reference throughout the specification to “one exemplary implementation”, “another exemplary implementation”, “an exemplary implementation”, and so forth, means that a particular element (e.g., feature, structure, and/or characteristic) described in connection with the exemplary implementation is included in at least one configuration described herein, and may or may not be present in other exemplary implementation(s). In addition, it is to be understood that the described elements may be combined in any suitable manner in the various exemplary implementation(s), and is provided as examples intended to provide an understanding of the disclosed technology.

The term “module” may refer to at least a self-contained component (unit, system or element) that is used in combination with other modules and/or components and/or a separate and distinct unit of hardware and/or software that may be used as a component in a device, such as a dermascope. The term “module” may also refer to at least a self-contained assembly of electronic components and circuitry, such as a circuit configured to control the actuator, wavelength, intensity and duration of an LED light that is installed as the RS unit or the imaging module. The term “module” may be used interchangeably with the term “unit.”

In the context of the disclosure, the term “operable” means the system and/or the device and/or the program, or a certain element or step is fully functional, sized, adapted and calibrated, comprises elements for, and meets applicable operability requirements to perform a recited function when activated, coupled, implemented, actuated, effected, realized, or when an executable program is executed by at least one processor associated with the system and/or the device. In relation to systems and circuits, the term “operable” means the system and/or the circuit is fully functional and calibrated, comprises logic for, having the hardware and firmware necessary, as well as the circuitry for, and meets applicable operability requirements to perform a recited function when executed by at least one processor.

Accordingly and in an exemplary implementation, provided herein is a system for in situ characterizing irregularities of at least one of: a tissue surface, and a tissue volume in a patient in need thereof, comprising: a housing defining longitudinal axis and a proximal base with a transparent observation plate (TOP) for user's direct vision through lens and a transparent contact plate (TCP) for positioning over a portion of the at least one of: the tissue surface, and the tissue volume; at least one radiation source (RS) unit operably coupled to the housing or to a rotating annulus disposed distal to the proximal base, the at least one RS sized and configured to illuminate in a variable manner the portion of the at least one of: the tissue surface, and the tissue volume, at a predetermined angles relative to the longitudinal axis (Z_{L}) of the housing; a lens disposed distal to the at least one RS unit; an imaging module disposed distal to the at least one RS unit, sized and configured to capture a Temporal Shadow Image (TSI) above the at least one of: the tissue surface, and the tissue volume; and Optionally a polarizing or filter module disposed between the at least one RS unit and TCP. Optionally a polarizing or filter module disposed between the TCP and imaging module. Optionally a polarizing or filter module disposed between the TCP and TOP, wherein (i) the at least one RS unit is configured to illuminate one spectral band or multiple spectral bands, (ii) the at least one RS unit comprises at least one illuminator operably coupled to the housing, operable to vary at least one of: angle of illumination (PV), intensity, and spectrum [to generate TSIs to calculate LMB, SGPI and LPFI], wherein (iii) the at least one illuminator is configured to emit collimated light at an angle of about between 0° and about 20°, (iv) the at least one RS unit is coupled to a rotatable annulus, the rotatable annulus operable to perform at least one of: rotate around the longitudinal axis, and translate along the longitudinal axis relative to the portion of the at least one of: the tissue surface, and the tissue volume, (v) the rotatable annulus is operably coupled to an actuator, configured to selectively effect at least one of: annulus rotation, annulus translation relative to the portion of the at least one of: the tissue surface, and the tissue volume, along the longitudinal axis, wherein (vi) each illuminator separate from an adjacent illuminator by no less than 1°, wherein (vii) the TCP is coupled to the top of the housing, sized and configured to allow direct observation of the portion of the at least one of: the tissue surface, and the tissue volume, by a user, the system further comprising: a prismatic element, a mirror or partial mirror, sized and configured to divert light that returns back from the skin lesion to TOP and the imaging module, wherein the system (viii) further comprising: a communication module configured to maintain communication with a communication network; and a CPM, in communication with the imaging module, the at least one illuminator, the actuator, and the communication module, the CPM further comprises at least one processor in communication with a non-transitory memory storage device storing thereon a processor-readable media with a set of executable instructions, configured when executed, to cause the at least one processor to: using the plurality of illuminators, illuminate the lesion from a plurality state of a PV; capture an image at each PV state, each image configured to provide a single TSI, thereby providing a plurality of TSI's; using the captured plurality of TSI's, generate LMB images and a cumulative SGPI and LPFI, as well as (ix) a communication module configured to maintain communication with a communication network; and a CPM, in communication with the imaging module, the at least one illuminator, the actuator, and the communication module, the CPM further comprises at least one processor in communication with a non-transitory memory storage device storing thereon a processor-readable media with a set of executable instructions, configured when executed, to cause the at least one processor to: actuate the actuator to rotate the annulus; while rotating, capture a plurality of images from the imaging module, each image providing a single TSI; using the captured plurality of TSI's, generate LMB images, SGPI and LPFI, wherein (x) at least one of: the predetermined angles of at least one illuminator, and the distance between the at least one RS unit and the portion of the at least one of: the tissue surface, and the tissue volume, is selectively variable and wherein (xi) the portion of the at least one of: the tissue surface, and the tissue volume, is a skin lesion.

In another exemplary implementation, provided herein is a method of imaging and characterizing SViR of a portion of the at least one of: a tissue surface, and a tissue volume, of a patient in need thereof, implementable in a system comprising: a housing defining longitudinal axis and a proximal base with a TCP for positioning over at least a portion of the skin lesion of the patient; at least one RS unit operable to provide a plurality PV states of illuminators each operably coupled to the housing, disposed distal to the proximal base, sized and configured to illuminate in a variable manner at a predetermined illumination angles around the longitudinal axis of the housing; a lens disposed distal to the at least one RS unit; an imaging module sized and configured to capture TSIs for each PV state of the at least one RS unit; and optionally a polarizing or filter module disposed between the RS unit and the skin and/or between the skin and the lens at TCP and/or imaging module, the method comprising: positioning the TCP over the portion of the at least one of: the tissue surface, and the tissue volume; using the at least one RS unit at a plurality PV states, illuminate the portion of the at least one of: the tissue surface, and the tissue volume; capturing an image at each PV state, each image providing a single TSI; and using the captured TSI's, generating LMB images, SGPI and LPFI of the portion of the at least one of: the tissue surface, and the tissue volume, representing the SViR, wherein (xii) each illuminator is tilted at a predetermined angle, the angle being selectively variable and configured to collimate the radiation on the same area, and (xiii) is configured to collimate the emitted light to an angle of between 0° and about 20°, wherein (xiv) the system further comprises a rotatable annulus operably coupled to the housing, wherein the rotatable annulus is additionally operably coupled to an actuator, configured to selectively radially rotate the annulus and axially translate the rotatable annulus along the housing longitudinal axis, the method further comprising (xv) capturing a plurality of images at different axial distance from the portion of the at least one of: the tissue surface, and the tissue volume, wherein (xvi) the plurality of illuminators are radially coupled to the housing, each illuminator configured to illuminate at a predetermined timing configuration, and wherein the timing configuration configured to illuminate the lesion from varying radial angles, the system further comprising (xvii) a TCP coupled to the top of the housing, sized and configured to allow direct observation of the skin lesion by users; and a prismatic element, mirror or partial mirror, sized and configured to divert light that returns back from the skin lesion to the lenses, as well as (xviii) a communication module configured to maintain communication with a communication network, and a CPM, in communication with the imaging module, the at least one illuminator, the actuator, and the communication module, the CPM further comprises at least one processor in communication with a non-transitory memory storage device storing thereon a processor-readable media with a set of executable instructions, the method further comprising: actuating the actuator to at least one of: rotate the annulus arbitrarily, translate the annulus relative to the portion of the at least one of: the tissue surface, and the tissue volume, and vary the angle of the at least one illuminator; capturing a plurality of TSI's from the imaging module; using the captured plurality of TSI's, generating LMB images, SGPI and LPFI of the portion of the at least one of: the tissue surface, and the tissue volume, representing the SViR, wherein (xix) the step of generating the LMB images, SGPI and LPFI of the portion of the at least one of: the tissue surface, and the tissue volume, comprises capturing and rendering time-variant phase appearance image of the portion of the at least one of: the tissue surface, and the tissue volume, comprising (xx) comprises: extracting the phase of repeated signal or full period of the annular in each pixel of the camera; generating a plurality of images, each image being of a single image per PV state; and combining the plurality of images to form single image of phase map after bias manipulation to generate LMB, then SGPI and LPFI, wherein (xxi) the system further comprises a communication module configured to maintain communication with a communication network, and a CPM, in communication with the imaging module, the at least one illuminator, the actuator, and the communication module, the CPM further comprises at least one processor in communication with a non-transitory memory storage device storing thereon a processor-readable media with a set of executable instructions, the method further comprising: using the plurality of illuminators, illuminating the lesion from a plurality of radial PV's; capturing an image at each PV state, each image forms a single TSI; using the captured plurality of TSI's, generating the LMB, SGPI and LPFI, (xxii) further comprising comparing the LMB and/or a single image generated by SGPI and/or LPFI referring the portion of the at least one of: the tissue surface, and the tissue volume, obtained from the same patient on another occasion, and wherein (xxiii) the portion of the at least one of: the tissue surface, and the tissue volume, is a skin lesion.

While particular exemplary implementation(s) have been described, alternatives, modifications, variations, improvements, and substantial equivalents that are or may be presently unforeseen may arise to applicants or others skilled in the art. Accordingly, the appended claims as filed and as they may be amended, are intended to embrace all such alternatives, modifications variations, improvements, and substantial equivalents.

1.-25. (canceled)

26. A system for in situ characterizing surface/volume irregularities (SViR) of a sample or tissue, comprising:
a. a housing defining longitudinal axis and comprising a transparent contact plate (TCP) for positioning over a portion of said sample or tissue;
b. at least one radiation source (RS) unit operably coupled to the housing and configured to be selectively rotatable about said longitudinal axis, the at least one RS being sized and configured to illuminate said sample or tissue in a variable manner at one or more predetermined angles relative to the longitudinal axis of the housing;
c. one or more lenses disposed distal to the at least one RS unit;
d. an imaging module disposed distal to the TCP, sized and configured to capture a Temporal Shadow Image (TSI) above the sample or tissue.

27. The system of claim 26, further comprising at least one polarizer filter positioned at one or more selected locations selected from between said RS unit and said TCP, between said TCP and said imaging module.

28. The system of claim 26, wherein the at least one RS unit comprises at least one illuminator operably coupled to the housing, operable to selectively vary at least one of: angle, intensity, and spectrum of illumination.

29. The system of claim 26, wherein said RS unit is configured to emit a generally collimated light having divergence angle not exceeding 20°.

30. The system of claim 26, wherein the at least one RS unit is coupled to a rotatable annulus, the rotatable annulus is operable to selectively perform at least one of: rotation around the longitudinal axis, and translation along the longitudinal axis relative to location of the TCP.

31. The system of claim 31, wherein the rotatable annulus is operably coupled to an actuator, configured to selectively effect at least one of: annulus rotation, annulus translation relative to the TCP along the longitudinal axis.

32. The system of claim 26, further comprising:
a. a communication module configured to maintain communication with a communication network; and
b. a central processing module (CPM), in communication with the imaging module, the at least one illuminator, the actuator, and the communication module, the CPM further comprises at least one processor in communication with a non-transitory memory storage device storing thereon a processor-readable media with a set of executable instructions, configured when executed, to cause the at least one processor to:
i. operating the RS unit to illuminate the sample or tissue using a plurality of Poynting Vector (PV) states;
ii. capturing an image at each of said plurality of PV states, each image configured to provide a single TSI, thereby providing a plurality of TSI's;
iii. using the captured plurality of TSI's to generate Lateral Motion of Backscattering Images (LMBI), cumulative Shadow Gradient Pattern Image (SGPI) and Lateral Phase Flow Image (LPFI).

33. The system of claim 26, further comprising:
a. a communication module configured to maintain communication with a communication network; and
b. a central processing module (CPM), in communication with the imaging module, the at least one illuminator, the actuator, and the communication module, the CPM further comprises at least one processor in communication with a non-transitory memory storage device storing thereon a processor-readable media with a set of executable instructions, configured when executed, to cause the at least one processor to:
i. actuate the actuator to rotate the annulus;
ii. while rotating, capture a plurality of images from the imaging module, each image providing a single TSI;
iii. using the captured plurality of TSI's, generate at least one of Lateral Motion of Backscattered Light images (LMBI), Shadow Gradient Pattern Image (SGPI) and Lateral Phase Flow Image (LPFI).

34. A method for imaging and characterizing surface and/or volume irregularities (SViR) of a portion of a sample or tissue, the method comprising:
a. operating at least one radiation source (RS) unit for illuminating at least a portion of the sample or tissue using a plurality of Pointing vector (PV) states;
b. capturing at least one image for each of said plurality of PV states, each image providing a Temporal Shadow Image (TSI); and
c. using the captured TSI's and generating at least one of Lateral Motion of Backscattered Light Images (LMBI), Shadow Gradient Pattern Image (SGPI) and Lateral Phase Flow Image (LPFI) of the portion of the at least one of: the tissue surface, and the tissue volume, representing the SViR.

35. The method of claim 34, wherein said operating at least one RS comprises illuminating said sample or tissue using a selectively variable tilt angle, with at least one collimated radiation toward a surface of the sample or tissue.

36. The method of claim 34, wherein said operating at least one RS comprises providing illumination of said portion of said sample or tissue, with a selected number of angular orientations with respect to a longitudinal axis perpendicular to said portion of sample or tissue, and axial distances from said portion of sample or tissue.

37. The method of claim 34, further comprising: capturing a plurality of images associated with a plurality of RS different axial distances along said longitudinal axis from the sample or tissue.

38. The method of claim 34, further comprising:
a. illuminating said portion of sample or tissue through a transparent contact plate (TCP) configured to be position on said sample or tissue; and
b. collecting backscattered radiation reflected from said sample or tissue through said transparent contact plate using a prismatic element, mirror or partial mirror, configured to divert light that returns back from the skin lesion to the lenses.

39. The method of claim 34, further comprising rendering a time-variant phase appearance image of the portion of the at least one of: the tissue surface, and the tissue volume.

40. The method of claim 39, wherein said rendering time-variant phase appearance image of the portion of said sample or tissue, comprises:
a. extracting for each pixel of the camera a phase of a signal;
b. generating a plurality of images, each image corresponding to a single PV state; and
c. combining the plurality of images to form a phase map in order to generate said at least one of LMBI, SGPI and LPFI.

41. The method of claim 34, further comprises executing using one or more processors for:
a. generating operational instructions for illuminating the sample or tissue from a plurality of PV states using at least one illuminator;
b. generating operational instructions for capturing at least one image for each of said plurality of PV states, each image forming a single TSI;
c. processing the plurality of TSI's to generate said LMBI, SGPI and LPFI.

42. The method of claim 40, wherein said processing further comprises comparing a first LMBI, SGPI and LPFI of the sample or tissue, with a second LMBI, SGPI and LPFI of the portion of the sample or tissue, obtained from the same patient on one or more different occasions.

43. The system of claim 26, wherein said sample of tissue comprises a portion of at least one of: biological tissue, fruit or vegetable.

44. The method of claim 34, wherein said sample of tissue comprises a portion of at least one of: biological tissue, fruit or vegetable.
